(19) United States
(12) Patent Application Publication
(10) Pub. No.: US 2014/0171831 A1
(43) Pub. Date: Jun. 19, 2014

(54) Clear/Translucent Tube Fluids Flash Display with Receiving Hub (Vacufly)

(71) Applicants: Johnny Dieu, Los Angeles, CA (US); Dave Dieu, Los Angeles, CA (US)

(72) Inventors: Johnny Dieu, Los Angeles, CA (US); Dave Dieu, Los Angeles, CA (US)

(21) Appl. No.: 13/716,720

(22) Filed: Dec. 17, 2012

(51) Int. Cl. A61B 5/15

(52) U.S. Cl. CPC A61B 5/150732
USPC 600/576

(57) ABSTRACT

The invention at hand is a needle assembly device comprising a clear tube attachment with a receiving female twist on end for fluids display where it will show the user if a vein has been successfully punctured and allow easier blood draw, wherein the user could scale between different gauge needles and interchange the needles parts depending on the situation. Preferably, the tube is made of a polypropylene material whereby the fluids will display when a vein has successfully pierced.

This device is used specifically for blood drawing situations and will allow easier blood draws for blood tests and blood sampling. The purpose of the Vacufly is to have a multipurpose needle in which takes any gauge type needle needed for the blood draw while a 21 gauge needle is the standard. With this innovation blood draw can be detected with any attachable needle due to a flash seen in the tube when a vein has been punctured. In this specification of the blood draw device a wide tube is placed in the Vacufly device where if a vein is punctured venous blood will rush towards the tube a few centimeters long, by which will alert to the extractor that the needle has gone deep enough for easy blood draw. The needle's main innovation is a clear tube in which blood will flow through and alert the professional when the vein is punctured. Moreover the device is attached to a hub which is receives the twist on from the tube to secure blood flow.

The Vacufly is an innovation started by two brothers named Johnny Dieu and Duy Dieu who have been experienced in the medical profession for over the years. These two brothers are of Vietnamese heritage but are full-fledged United States citizens with the American dream. Living in atomic family around greater Los Angeles region near the Los Angeles river the family has been exposed to medical devices and medical situations over the past generations, having worked in the field for long enough the brothers found a breakthrough that may make patient blood drawing a lot easier for there are many “hard sticks” a medical professional may encompass when drawing patient blood. It is for that reason that the Vacufly was innovated in order to make patient and medical professional's life a lot easier.

### CROSS-REFERENCES TO RELATED APPLICATIONS

The Vacufly is a needle assembly device that is usually assembled together with a needle protection device in order to allow the collection of blood specimens from the device to a tube or many tubes depending on the situation, and is connected with a specially designed female receiver hub. This device therefore is a synergy with a needle guard in order to allow easy assembly of the tube from the needle for blood draw. US patent referenced patents to this invention can be found from:

| Cited | | |
|---|---|---|
| Patent | Issue date | Title |
| U.S. Pat. No. 1,779,451 | Oct. 28, 1930 | CHARLES SPOUSEI |
| U.S. Pat. No. 2,004,050 | Jun. 4, 1935 | HYPODERMIC NEEDLE PACKAGE |
| U.S. Pat. No. 2,700,385 | Jan. 25, 1955 | ORTIZ |
| U.S. Pat. No. 2,836,942 | Jun. 3, 1958 | METHOD OF ENCASING AND STERILIZING NEEDLES |
| U.S. Pat. No. 2,854,976 | Oct. 7, 1958 | PROTECTIVE DEVICE FOS NEEDLES OF |
| U.S. Pat. No. 2,953,243 | Sep. 20, 1960 | DISPOSABLE NEEDLE ASSEMBLY |
| U.S. Pat. No. 3,021,942 | Feb. 20, 1962 | NEEDLE PACKAGE |
| U.S. Pat. No. 3,073,307 | Jan. 15, 1963 | STEVENS |
| U.S. Pat. No. 3,074,542 | Jan. 22, 1963 | PACKAGE FOR HYPODERMIC NEEDLES |
| U.S. Pat. No. 3,255,873 | Jun. 14, 1966 | COMBINATION SEALING AND DISPENSING DEVICE |
| U.S. Pat. No. 3,294,231 | Dec. 27, 1966 | DENTAL NEEDLE SHIELD |
| U.S. Pat. No. 3,323,523 | Jun. 6, 1967 | INTRAVENOUS CATHETER ASSEMBLY WITH |
| | | DIVISIBLE NEEDLE SHEATH PORTIONS |
| U.S. Pat. No. 3,329,146 | Jul. 4, 1967 | NEEDLE CONTAINER |
| U.S. Pat. No. 3,333,682 | Aug. 1, 1967 | DISPOSABLE NEEDLE CONTAINER |
| U.S. Pat. No. 3,367,488 | Feb. 6, 1968 | HYPODERMIC SYRINGE PACKAGE |
| U.S. Pat. No. 3,382,865 | May 14, 1968 | DEVICE FOR TAKING MULTIPLE BLOOD SAMPLES OR |
| | | THE LIKE |
| U.S. Pat. No. 3,485,239 | Dec. 23, 1969 | SELF-CONTAINED STERILE SYRINGE |
| U.S. Pat. No. 3,537,452 | Nov. 3, 1970 | NEEDLE COVER AND BEVEL GUARD |
| U.S. Pat. No. 3,585,984 | 1971 | BLOOD TRANSFER DEVICE |
| U.S. Pat. No. 3,610,240 | Oct. 5, 1971 | INTRAVENOUS CATHETER APPARATUS WITH |
| U.S. Pat. No. 3,658,061 | 1972 | NEEDLE GUARD |
| U.S. Pat. No. 3,664,879 | May 23, 1972 | VENT PLUG FOR A FILLING OPENING OF A CELL OF |
| | | AN ELECTRIC STORAGE BATTERY |
| U.S. Pat. No. 3,817,240 | 1974 | USGOMM-DC |
| U.S. Pat. No. 3,828,775 | Aug. 1, 1974 | SELF-PACKAGED HYPODERMIC SYRINGE |
| U.S. Pat. No. 3,859,998 | 1975 | INTRAVENOUS NEEDLE ASSEMBLY |
| U.S. Pat. No. 3,874,367 | 1975 | VALVED BLOOD SAMPLING NEEDLE ASSEMBLY |
| U.S. Pat. No. 3,886,930 | 1975 | BLOOD COLLECTING ASSEMBLY |
| U.S. Pat. No. 3,890,971 | Jun. 2, 1975 | SAFETY SYRINGE |
| U.S. Pat. No. 3,904,033 | 1975 | PICK-GUARD |
| U.S. Pat. No. 3,934,722 | Jan. 27, 1976 | Sterile needle package |
| U.S. Pat. No. 3,968,876 | Jul. 13, 1976 | Sealed container with a sterilized hypodermic needle within it |
| | | and method for effecting the sealing thereof |
| U.S. Pat. No. 4,106,497 | Aug. 15, 1978 | Multiple sample needle assembly with indicator means |
| U.S. Pat. No. 4,108,175 | Aug. 22, 1978 | Catheter insertion apparatus |
| U.S. Pat. No. 4,113,090 | Sep. 12, 1978 | Medical instrument package |
| U.S. Pat. No. 4,139,009 | Feb. 13, 1979 | Hypodermic needle assembly with retractable needle cover |
| U.S. Pat. No. 4,154,229 | May 15, 1979 | Blood collection system with venipuncture indicator |
| U.S. Pat. No. 4,166,450 | Sep. 4, 1979 | Device and procedure for collecting a succession of intravenous |
| | | blood samples |
| U.S. Pat. No. 4,175,008 | Nov. 20, 1979 | Culture specimen collection and transport package |
| U.S. Pat. No. 4,193,400 | Mar. 18, 1980 | Intravenous needle assembly with air bleed plug |
| U.S. Pat. No. 4,207,870 | Jun. 17, 1980 | Blood sampling assembly having porous vent means vein entry |
| | | indicator |
| U.S. Pat. No. 4,269,186 | May 26, 1981 | Intravenous needle assembly with air bleed plug |
| U.S. Pat. No. 4,300,678 | Nov. 17, 1981 | Syringe package with evidence of opening |
| U.S. Pat. No. 4,312,362 | Jan. 26, 1982 | Single sample needle with vein entry indicator |
| U.S. Pat. No. 4,317,445 | Mar. 2, 1982 | Catheter insertion unit with separate flashback indication for the |
| | | cannula |
| U.S. Pat. No. 4,340,068 | Jul. 20, 1982 | Multiple sample needle with vein entry indicator |
| U.S. Pat. No. 4,375,849 | Mar. 8, 1983 | Syringe needle removal and disposal device |
| U.S. Pat. No. 4,398,544 | Aug. 16, 1983 | Single and multiple sample needle assembly with vein entry |
| | | indicator |
| U.S. Pat. No. 4,409,990 | Oct. 18, 1983 | Fluid sampling needle assembly and method of use thereof |
| U.S. Pat. No. 4,412,548 | Nov. 1, 1983 | Multiple sample needle assembly |
| U.S. Pat. No. 4,416,290 | Nov. 22, 1983 | Multiple sample needle assembly with vein indication |
| U.S. Pat. No. 4,416,291 | Nov. 22, 1983 | Multiple sample needle assembly with vein entry indicator |
| U.S. Pat. No. 4,418,703 | Dec. 6, 1983 | Multiple sample needle assembly |
| U.S. Pat. No. 4,430,082 | Feb. 7, 1984 | Hypodermic syringe assembly |
| U.S. Pat. No. 4,436,098 | Mar. 13, 1984 | Needle assembly with vein entry indicator |
| U.S. Pat. No. 4,444,203 | Apr. 24, 1984 | Intravenous catheter placing and specimen gathering device |
| U.S. Pat. No. 4,592,744 | Jun. 3, 1986 | Self-resheathing needle assembly |
| U.S. Pat. No. 4,634,428 | Jan. 6, 1987 | Cover for a disposable syringe |
| U.S. Pat. No. 4,643,722 | Feb. 17, 1987 | Closure system for storage, transport and disposal of |
| | | hypodermic needles |
| U.S. Pat. No. 4,659,330 | Apr. 21, 1987 | Hypodermic syringe needle guard |
| U.S. Pat. No. 4,664,249 | May 12, 1987 | Turn around device for filter cigarettes |
| U.S. Pat. No. 4,664,654 | May 12, 1987 | Automatic protracting and locking hypodermic needle guard |
| U.S. Pat. No. 4,671,408 | Jun. 9, 1987 | Temper-resistant protective capping device for filled syringes |
| U.S. Pat. No. 4,679,571 | Jul. 14, 1987 | Blood sample needle assembly with vein indicator |
| U.S. Pat. No. 4,681,567 | Jul. 21, 1987 | Syringe with safety sheath |
| U.S. Pat. No. 4,695,274 | Sep. 22, 1987 | Protected hypodermic needle |
| U.S. Pat. No. 4,702,738 | Oct. 27, 1987 | Disposable hypodermic syringe and needle combination having |
| | | retractable, accident preventing sheath |
| U.S. Pat. No. 4,723,943 | Feb. 9, 1988 | Sheathed syringe |
| U.S. Pat. No. 4,728,320 | Mar. 1, 1988 | Syringe cap with hammer |
| U.S. Pat. No. 4,728,321 | Mar. 1, 1988 | Syringe cap with adhesive holding plug |
| U.S. Pat. No. 4,731,059 | Mar. 15, 1988 | Combination needle shield/needle guard device positively |
| | | locked onto detachable needle assemblies for an evacuated |
| | | blood collection system and a hypodermic syringe |
| U.S. Pat. No. 4,735,311 | Apr. 5, 1988 | Needle shield assembly |
| U.S. Pat. No. 4,735,618 | Apr. 5, 1988 | Protective enclosure for hypodermic syringe |
| U.S. Pat. No. 4,737,144 | Apr. 12, 1988 | Syringe with selectively exposed and enveloped needle |
| U.S. Pat. No. 4,738,663 | Apr. 19, 1988 | Hypodermic needle shield |
| U.S. Pat. No. 4,743,233 | May 10, 1988 | Safety cap syringe |
| U.S. Pat. No. 4,746,008 | May 24, 1988 | Child-resistant box for storage of hazardous materials |
| U.S. Pat. No. 4,747,836 | May 31, 1988 | Needle guard, and assembly |
| U.S. Pat. No. 4,747,837 | May 31, 1988 | Syringe needle recapping protective device |
| U.S. Pat. No. 4,772,272 | Sep. 20, 1988 | Needle protective sleeve |
| U.S. Pat. No. 4,778,453 | Oct. 18, 1988 | Medical device |
| U.S. Pat. No. 4,781,697 | Nov. 1, 1988 | Removable protective shield for needle sheath |
| U.S. Pat. No. 4,782,841 | Nov. 8, 1988 | Medical device |
| U.S. Pat. No. 4,788,986 | Dec. 6, 1988 | Holder for blood collecting needle |
| U.S. Pat. No. 4,790,828 | Dec. 13, 1988 | Self-capping needle assembly |
| U.S. Pat. No. 4,793,484 | Dec. 27, 1988 | Box-shaped container for articles |
| U.S. Pat. No. 4,795,432 | Jan. 3, 1989 | Shield assembly for hypodermic injection devices |
| U.S. Pat. No. 4,795,443 | Jan. 3, 1989 | Syringe sealing device and method |
| U.S. Pat. No. 4,801,295 | Jan. 31, 1989 | Disposable hypodermic syringe and needle combination having |
| | | retractable, accident preventing sheath |
| U.S. Pat. No. 4,804,372 | Feb. 14, 1989 | Protective sheath for hypodermic needle |
| U.S. Pat. No. 4,813,426 | Mar. 21, 1989 | Shielded safety syringe having a retractable needle |
| U.S. Pat. No. 4,816,022 | Mar. 28, 1989 | Hypodermic syringe with sliding cap |
| U.S. Pat. No. 4,816,024 | Mar. 28, 1989 | Medical device |
| U.S. Pat. No. 4,819,659 | Apr. 11, 1989 | Blood withdrawal device with movable needle guard member |
| U.S. Pat. No. 4,820,277 | Apr. 11, 1989 | Safety cover for syringe needles |
| U.S. Pat. No. 4,826,490 | May 2, 1989 | Safety device for hypodermic needle or the like |
| U.S. Pat. No. 4,826,491 | May 2, 1989 | Needle bearing medical device with three-position shield |
| U.S. Pat. No. 4,838,871 | Jun. 13, 1989 | Needle guard, and assembly |
| U.S. Pat. No. 4,842,587 | Jun. 27, 1989 | No-prick hypodermic syringe |
| U.S. Pat. No. 4,844,089 | Jul. 4, 1989 | Needle for multiple vacuum blood sample devices |
| U.S. Pat. No. 4,846,796 | Jul. 11, 1989 | Protective system for safe disposition of a syringe and |
| | | hypodermic injection device carried thereon |
| U.S. Pat. No. 4,850,968 | Jul. 25, 1989 | Self-blocking hypodermic syringe for once-only use, |
| | | comprising a needle protection cap |
| U.S. Pat. No. 4,850,976 | Jul. 25, 1989 | Combination sheath and foldable shield for hypodermic syringe |
| | | needle |
| U.S. Pat. No. 4,850,977 | Jul. 25, 1989 | Button activated automatic needle sheath for disposable syringe |
| U.S. Pat. No. 4,850,978 | Jul. 25, 1989 | Drug delivery cartridge with protective cover |
| U.S. Pat. No. 4,850,994 | Jul. 25, 1989 | Hypodermic syringe |
| U.S. Pat. No. 4,850,996 | Jul. 25, 1989 | Safety needle |
| U.S. Pat. No. 4,858,607 | Aug. 22, 1989 | Plastic device for injection and obtaining blood samples |
| U.S. Pat. No. 4,863,434 | Sep. 5, 1989 | Automatic needle sheath for disposable syringe |
| U.S. Pat. No. 4,863,435 | Sep. 5, 1989 | Safety hypodermic syringe |
| U.S. Pat. No. 4,863,436 | Sep. 5, 1989 | Hypodermic needle with protective cover |
| U.S. Pat. No. 4,865,592 | Sep. 12, 1989 | Container and needle assembly |
| U.S. Pat. No. 4,867,746 | Sep. 19, 1989 | Needle shield |
| U.S. Pat. No. 4,872,552 | Oct. 10, 1989 | Safety packaging for hypodermic syringes with needles and the |
| | | like |
| U.S. Pat. No. 4,874,383 | Oct. 17, 1989 | Syringe shield |
| U.S. Pat. No. 4,874,384 | Oct. 17, 1989 | Needle safety guard |
| U.S. Pat. No. 4,883,469 | Nov. 28, 1989 | Guard assembly for hypodermic needle |
| U.S. Pat. No. 4,886,072 | Dec. 12, 1989 | Multiple sample needle assembly with vein indicator |
| U.S. Pat. No. 4,886,503 | Dec. 12, 1989 | Disposable covered needle for syringe |
| U.S. Pat. No. 4,888,001 | Dec. 19, 1989 | Cover for a disposable hypodermic needle |
| U.S. Pat. No. 4,892,107 | Jan. 9, 1990 | Single use, safety blood collection device |
| U.S. Pat. No. 4,892,521 | Jan. 9, 1990 | Protective cover for hypodermic needle |
| U.S. Pat. No. 4,894,052 | Jan. 16, 1990 | Flash detection in an over the needle catheter with a restricted |
| | | needle bore |
| U.S. Pat. No. 4,900,309 | Feb. 13, 1990 | Needle shield |
| U.S. Pat. No. 4,909,791 | Mar. 20, 1990 | Safety cover for syringe needles |
| U.S. Pat. No. 4,909,792 | Mar. 20, 1990 | Safety cover for syringe needles |
| U.S. Pat. No. 4,921,096 | May 1, 1990 | Package assembly |
| U.S. Pat. No. 4,927,018 | May 22, 1990 | Disposable covered needle safety assembly |
| U.S. Pat. No. 4,944,397 | Jul. 31, 1990 | Disposable covered needle for syringe |
| U.S. Pat. No. 4,966,591 | Oct. 30, 1990 | Needle assembly |
| U.S. Pat. No. 4,971,068 | Nov. 20, 1990 | Blood vessel locating needle assembly with thermochromic |
| | | indicator |
| U.S. Pat. No. 4,976,699 | Dec. 11, 1990 | Needle and safety cover assembly for syringes and the like |
| U.S. Pat. No. 4,982,842 | Jan. 8, 1991 | Safety needle container |
| U.S. Pat. No. 5,011,475 | Apr. 30, 1991 | Protector for intravenous and syringe needles |
| U.S. Pat. No. 5,011,479 | Apr. 30, 1991 | Cover and connector for hypodermic needle |
| U.S. Pat. No. 5,030,207 | Jul. 9, 1991 | Instantaneous vein entry indicator for intravenous needle |
| U.S. Pat. No. 5,032,116 | Jul. 16, 1991 | Flashback plug |
| U.S. Pat. No. 5,033,476 | Jul. 23, 1991 | Blood collecting tube |
| U.S. Pat. No. 5,055,102 | Oct. 8, 1991 | Swing-away disposable syringe needle cover |
| U.S. Pat. No. 5,069,225 | Dec. 3, 1991 | Blood collection and/or injection device and double-ended |
| | | medical needle and holder therefor |
| U.S. Pat. No. 5,078,693 | Jan. 7, 1992 | Safety hypodermic syringe |
| U.S. Pat. No. 5,078,698 | Jan. 7, 1992 | Axial eject hypodermic syringe holder |
| U.S. Pat. No. 5,092,845 | Mar. 3, 1992 | Catheter with needle gasket |
| U.S. Pat. No. 5,112,327 | May 12, 1992 | Double-sided needle assembly |
| U.S. Pat. No. 5,116,325 | May 26, 1992 | Needle assembly |
| U.S. Pat. No. 5,120,319 | Jun. 9, 1992 | Flash tube for intravenous catheter |
| U.S. Pat. No. 5,122,121 | Jun. 16, 1992 | Safety needle assembly |
| U.S. Pat. No. 5,135,509 | Aug. 4, 1992 | Hypodermic syringe |
| U.S. Pat. No. 5,137,518 | Aug. 11, 1992 | Instantaneous vein entry indicator for intravenous needle |
| U.S. Pat. No. 5,139,489 | Aug. 18, 1992 | Needle protection device |
| U.S. Pat. No. 5,151,089 | Sep. 29, 1992 | Retractable protective needle sheath |
| U.S. Pat. No. 5,154,285 | Oct. 13, 1992 | Needle assembly holder with rotatable safety sheath member |
| U.S. Pat. No. 5,181,523 | Jan. 26, 1993 | Blood sampling device with blood-viewing chamber |
| U.S. Pat. No. 5,188,611 | Feb. 23, 1993 | Safety sheath for needles, sharp instruments and tools |
| U.S. Pat. No. 5,197,954 | Mar. 30, 1993 | Hypodermic syringe having folding needle |
| U.S. Pat. No. 5,201,794 | Apr. 13, 1993 | Method for sampling blood specimen |
| U.S. Pat. No. 5,207,653 | May 4, 1993 | Safety needle and cap combination device |
| U.S. Pat. No. 5,217,025 | Jun. 8, 1993 | Blood collection and/or injection device and double-ended |
| | | medical needle and holder therefor |
| U.S. Pat. No. 5,222,502 | Jun. 29, 1993 | Blood collecting needle |
| U.S. Pat. No. 5,232,454 | Aug. 3, 1993 | Safety needle container |
| U.S. Pat. No. 5,232,455 | Aug. 3, 1993 | Syringe with protective housing |
| U.S. Pat. No. 5,242,411 | Sep. 7, 1993 | Piercing needle having hydrophobic filter vent |
| U.S. Pat. No. 5,242,417 | Sep. 7, 1993 | Self closing hinged syringe guard |
| U.S. Pat. No. 5,277,311 | Jan. 11, 1994 | Needle assembly holder with rotatable safety sheath member |
| | | and method of effecting proper alignment of a cannula using |
| | | such needle assembly holder |
| U.S. Pat. No. 5,290,246 | Mar. 1, 1994 | Piercing needle |
| U.S. Pat. No. 5,295,969 | Mar. 22, 1994 | Vascular access device with air-tight blood containment |
| | | capability |
| U.S. Pat. No. 5,303,713 | Apr. 19, 1994 | Blood collecting needle |
| U.S. Pat. No. 5,306,259 | Apr. 26, 1994 | Vascular access needle having an extended length body |
| U.S. Pat. No. 5,312,369 | May 17, 1994 | Safety hood for hypodermic needles |
| U.S. Pat. No. 5,401,251 | Mar. 28, 1995 | Safe cap covered injection system |
| U.S. Pat. No. 5,405,332 | Apr. 11, 1995 | Shield apparatus for syringe needle |
| U.S. Pat. No. 5,423,765 | Jun. 13, 1995 | Removable safety needle sheath |
| U.S. Pat. No. 5,450,856 | Sep. 19, 1995 | Phlebotomy needle attachable to a vacuum container with a |
| | | vent to preclude blood flashback |
| U.S. Pat. No. 5,462,534 | Oct. 31, 1995 | Needle assembly for use with a syringe |
| U.S. Pat. No. 5,485,854 | Jan. 23, 1996 | Safety blood collection tube holder and assembly therefor |
| U.S. Pat. No. 5,486,163 | Jan. 23, 1996 | Protective shield for hypodermic syringe |
| U.S. Pat. No. 5,490,841 | Feb. 13, 1996 | Safety sheath device |
| U.S. Pat. No. 5,496,281 | Mar. 5, 1996 | Spinal cannula with transparent grip part |
| U.S. Pat. No. 5,509,907 | Apr. 23, 1996 | Syringe needle guard assembly |
| U.S. Pat. No. 5,520,193 | May 28, 1996 | Blood collecting needle with vein indicator |
| U.S. Pat. No. 5,533,984 | Jul. 9, 1996 | Protection device for a body-penetrating syringe needle |
| U.S. Pat. No. 5,542,932 | Aug. 6, 1996 | Bloodless flashback vent |
| U.S. Pat. No. 5,584,816 | Dec. 17, 1996 | Hardpack shield for a pivoting needle guard |
| U.S. Pat. No. 5,599,313 | Feb. 4, 1997 | Needle shield assembly having safety indication features |
| U.S. Pat. No. 5,599,318 | Feb. 4, 1997 | Needle shield assembly having a releasable lock |
| U.S. Pat. No. 5,603,699 | Feb. 18, 1997 | Needle guard assembly |
| U.S. Pat. No. 5,632,732 | May 27, 1997 | Needle assembly having single handedly activated shield |
| U.S. Pat. No. 5,643,219 | Jul. 1, 1997 | Shielded needle assembly |
| U.S. Pat. No. 5,662,617 | Sep. 2, 1997 | Manually pivoted barrier assembly for piercing element |
| U.S. Pat. No. 5,665,075 | Sep. 9, 1997 | Method of making a needle shield assembly |
| U.S. Pat. No. 5,669,889 | Sep. 23, 1997 | Needle shield assembly having a single-use lock |
| U.S. Pat. No. 5,693,022 | Dec. 2, 1997 | Protective shield for hypodermic syringe |
| U.S. Pat. No. 5,697,914 | Dec. 16, 1997 | Control forward/flashback forward one hand introducer needle |
| | | and catheter assembly |
| U.S. Pat. No. 5,702,369 | Dec. 30, 1997 | Extendable device for enclosing cutting surfaces of surgical |
| | | instruments |
| U.S. Pat. No. 5,733,265 | Mar. 31, 1998 | Shielded needle assembly |
| U.S. Pat. No. 5,755,701 | May 26, 1998 | Blood withdrawal device |
| U.S. Pat. No. 5,807,351 | Sep. 15, 1998 | Protection device for sharp objects |
| U.S. Pat. No. 5,830,190 | Nov. 3, 1998 | Protected needle catheter placement device having needle |
| | | placement visualization features and method for its use |
| U.S. Pat. No. 5,836,920 | Nov. 17, 1998 | Needle guard |
| U.S. Pat. No. 5,885,249 | Mar. 23, 1999 | Syringe with cap |
| U.S. Pat. No. 5,893,844 | Apr. 13, 1999 | Indwelling needle set |
| U.S. Pat. No. 5,913,846 | Jun. 22, 1999 | Shielded needle assembly |
| U.S. Pat. No. 5,984,895 | Nov. 16, 1999 | Vascular blood flashback containment device with improved |
| | | sealing capability |
| U.S. Pat. No. 5,993,426 | Nov. 30, 1999 | Fluid absorbable needle sheath |
| U.S. Pat. No. 6,059,737 | May 9, 2000 | Adhesive gasket for blood collection needles |
| U.S. Pat. No. 6,077,244 | Jun. 20, 2000 | Catheter insertion device with retractable needle |
| U.S. Pat. No. 6,077,253 | Jun. 20, 2000 | Safety needle assembly |
| U.S. Pat. No. 6,080,137 | Jun. 27, 2000 | Needle protector |
| U.S. Pat. No. 6,096,006 | Aug. 1, 2000 | Blood-collection needle-disposal system |
| U.S. Pat. No. 6,110,160 | Aug. 29, 2000 | Blood extraction device having a holder and a sharpened-end |
| | | cannula |
| U.S. Pat. No. 6,120,482 | Sep. 19, 2000 | Pivotable guard for shielding a needle |
| U.S. Pat. No. 6,156,010 | Dec. 5, 2000 | Method and apparatus for introducing an intravenous catheter |
| U.S. Pat. No. 6,190,370 | Feb. 20, 2001 | Devices, systems and methods for determining proper |
| | | placement of epidural catheters |
| U.S. Pat. No. 6,261,263 | Jul. 17, 2001 | Hub of arterial puncture needle |
| U.S. Pat. No. 6,511,439 | Jan. 28, 2003 | Blood-collecting device |
| U.S. Pat. No. 6,533,760 | Mar. 18, 2003 | Flashback blood collection needle |
| U.S. Pat. No. 6,964,658 | Nov. 15, 2005 | System and method for facilitating hemostasis of blood vessel |
| | | punctures with absorbable sponge |
| U.S. Pat. No. 7,160,267 | Jan. 9, 2007 | Blood drawing device with flash detection |
| USRE31086 | Nov. 23, 1982 | Plastic hinge construction |

### BACKGROUND OF INVENTION

1. Field of Invention

The invention at hand is a clear hardened clear and or translucent attachment between the needle and the needle's hub, this clear see through attachment will show the blood flow the of the patient and allow the extractor to collect blood much more easier due to the flash of blood when a vein has been punctured. The tube is clearly attached the intravenous end of the needle and is twisted onto a designed female hub which receives the attachment tube. It solves the problem of inserting the needle into the patient and knowing if the vein is hit or not due to the clear tube or translucent in between the needle and hub.

2. Description of Related Art

The typical needle assembly device of this design is usually used with the conjunction with a needle holder therefore being able to collect blood specimens into tubes for blood collection safely. With the proper twist on needle holder the chance of premature disengagement. An established designed needle protector would be best for twisting on the needle to avoid needle injuries.

### SUMMARY OF INVENTION

The invention is a clear tube that allows blood to be seen when a vein has been punctured by the needle and is also detachable from the clear twist on to the hub in order to interchange the typical 21 and 23 gauge needles.

The advantage of a clear tube attached to this device is that it would still allow the blood flow from the patient to the vacuum tube to be fluid as any other needle extraction device yet would allow the user to view through the clear tube the blood flow after the vein is hit. This all in all removes the guess work behind whether or not the vein has been hit and would therefore save countless vacuum tubes and blood extraction attempts due to vein misreads and needle overextension when a vein has completely been pierced through.

On another note the twist off function in changing between different gauge needles adds a duality to this device where using a smaller gauge needle such as the 23 on geriatrics and children where veins are hard to extract blood from. Both the 23 and 21 gauge needles will have the clear tube in order to view if blood has been struck in the patient, but the 21 gauge needle will be the standard use needle for the device for a 21 gauge needle is used generally on everyday patients. The 23 gauge needle attachment will come as an add-on in case the 21 gauge needle would be difficult for blood extraction.

A feature to notice is that this device is safe to use as with any other vacutainer type blood extraction device. A conventional needle protector could be attached to the side to cap the needle after work has been done to avoid needle-sticks to the patient or professional in charge with extraction. It is a design where a longer cap is needed to accommodate the extra length of the needle due to the tempered clear tube. Any needle cap lock device that locks after capping would be advantages to the professional to avoid catching blood borne pathogens.

### DESCRIPTION OF DRAWINGS

FIG. 1 is a perspective view of a needle assembly or Vacufly device with a plastic tube attachment to the needle between the needle and its flange.

FIG. 2 is a perspective view of a needle assembly with a needle protector being put together.

FIG. 3 is a perspective view of the needle being capped by longer safety cap device.

FIG. 4 is a perspective cross section view of the Vacufly.

### DETAILED DESCRIPTIONS

The said invention while fulfilling many embodiments in different forms, in the drawings the suggested embodiments of the invention, with knowledge of the device and its counterparts is considered exemplary of the principles of the invention and is not intended to limit or undermine the invention to the construction illustrated. Further modifications or models will be evident to and readily made by those skilled in the art without straying from the scope and schematics of said invention. The scale of this invention therefore will be supplemented by the relevant claims

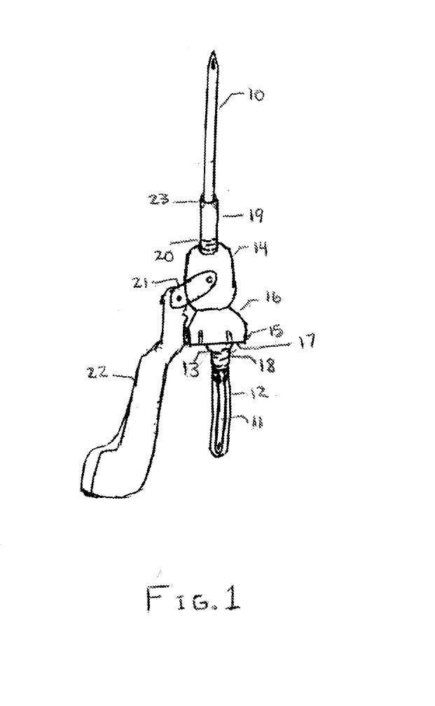

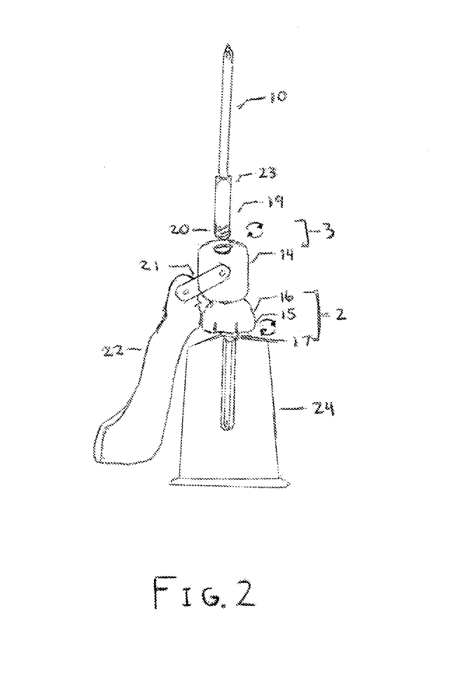

Referring to the drawings references, the reference is classified as characters and refers to the like parts through several views thereof; FIGS. 1 through 4 illustrate the needle assembly with the needle attachment to the flange. In assembly with all connected parts, FIG. 2 demonstrates a fully connected blood draw device for blood extraction purposes, and this consists of a needle, the tube, and hub.

As shown in FIGS. 1, 2, and 3; these drawings include the needle with a non-patient end 11, and an intravenous end 10 and also a passageway in-between the non-patient end and the intravenous end. An elastomeric sleeve 12 covers the non-patient end for vacuum tube collection.

Shown in FIGS. 1, 2, and 3, the hub includes a threaded end 13, a ribbed end 14 and a passageway extending between the threaded end and the oval end. Threaded end 13 and oval end 14 are separated by the flange 15. Flange 15 includes a threaded end surface 17 and an oval end surface 16. The threaded end 13 extends a non-patient end needle 10, and the intravenous end 10 extends off the oval end 14. In preference, the threaded end 13 composed of male threads 18 for mounting the connection between the hub and a conventional needle holder. Moreover, on the oval end 14 comprises of a female receiving attachment to twist on the male end clear tube attachment 20 of the clear tube 19 that is kept tightly connected with an elastic and or solid material 23. Attached to the side is the hinge 21 that connects to a needle cap 22 that safely caps the needle after the procedure.

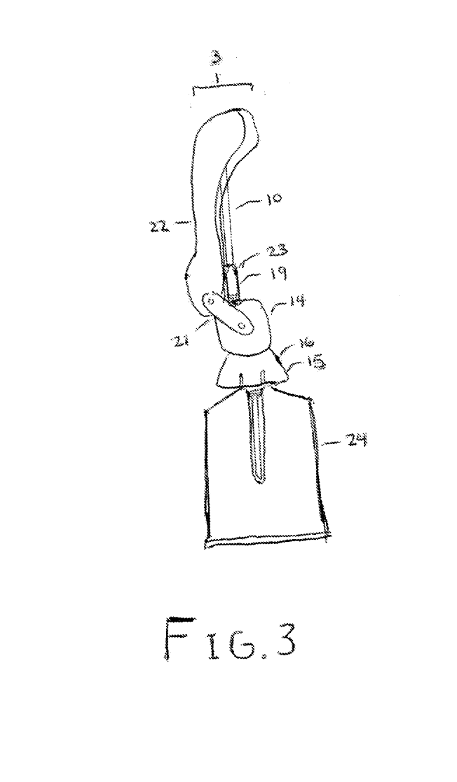

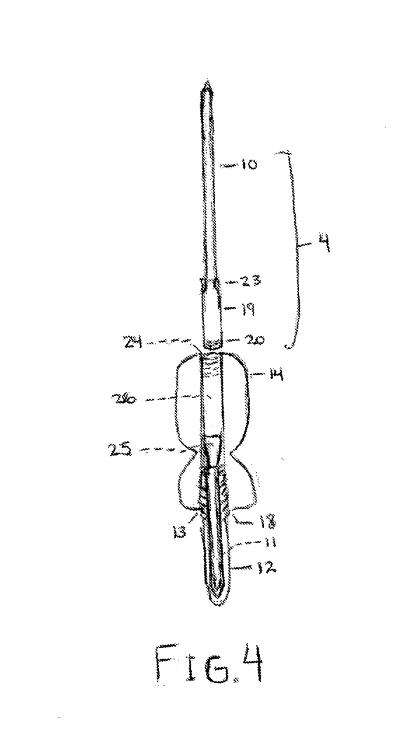

In FIG. 2 the needle assembly is attached to a generic needle protector used in most assemblies with said needle 2. The needle 10 and clear tube attachment 19 is being assembled in 3 and is ready for use afterwards. FIG. 3 comprises of a fully attached device with a needle cap fully capping the needle 10. FIG. 4 shows the needle attachment 4 which comprises of the needle 10, clear elastic or solid connector 23, the clear tube and the male twist on end 20. The oval end contains the following 26 female end clear tube receptor 24, fluids pathway 26 and funnels out in the funnel 25 to the needle end 11. The funnel 25 is connected to the needle 11 via adhesive material 26.

What is claimed:

1. A assemble comprising of:
A needle connected to a said hub made of a non-patient end and an intravenous end;
A hub comprising a threaded end, a ribbed or oval end, and a flange separating the threaded end and ribbed end comprising a threaded end surface and a ribbed end surface.

2. The assembly of claim 1, wherein said clear tube is in between the needle and flange is made of a material made of a hard and clear plastic tube.

3. The assembly of claim 2, wherein said assembly device uses the clear tube as any mixture of glass and plastic material.

4. The assembly of claim 3, wherein said assembly device uses the clear tube made of a tempered plastic rubber material.

5. The assembly of claim 4, wherein said assembly devices uses a clear tube made of a hardened clear rubber material.

6. The assembly of claim 5, wherein said assembly devices uses a clear tube made of a glass material.

7. The assembly of claim 6, wherein said assembly devices uses a polypropylene tube material to construct the tube.

8. The assembly of claim 7, wherein said assembly devices uses silicon tube material to construct the tube.

9. An assembly comprising:
A hub comprising of a threaded end, a flange separating a threaded end between the threaded end and ribbed or oval end comprising of a threaded end surface and a ribbed or oval end surface;
A needle connected to said hub made a non-patient end and an intravenous end;
A collar connected to said hub and a shield movably connected to a said collar,
A needle holder,
A clear tube located on between the said oval or ribbed end surface and flange, whereby said attachable tube forms an attachable secure connection between said hub of said needle assembly and a needle holder.

10. The assembly of claim 9, wherein said assembly device is in between the needle and flange is made of a material made of a hard and clear plastic tube.

11. The assembly of claim 10, wherein said assembly device uses the clear tube as any mixture of glass and plastic material.

12. The assembly of claim 11, wherein said assembly device uses the clear tube made of a tempered plastic rubber material.

13. The assembly of claim 12, wherein said assembly devices uses a clear tube made of a hardened clear rubber material.

14. The assembly of claim 13, wherein said assembly devices uses a clear tube made of a glass material.

15. The assembly of claim 14, wherein said assembly devices uses a polypropylene tube material to construct the tube.

16. The assembly of claim 15, wherein said assembly devices uses a polypropylene tube material to construct the tube.

17. The drawings of the Vacufly are not exact and the proportions of parts are not limited to the drawings of the product, therein the size and scale of the product are indefinite.
